(19) United States
(12) Patent Application Publication
(10) Pub. No.: US 2007/0026477 A1
(43) Pub. Date: Feb. 1, 2007

(54) Whole cell assay involving cathepsin s

(76) Inventors: Jean-Pierre Falgueyret, Rigaub (CA); Christophe Mellon, L'lle Bizard (CA)

Correspondence Address: MERCK AND CO., INC, P O BOX 2000, RAHWAY, NJ, 07065-0907, US

(21) Appl. No.: 10/571,016

(22) PCT Filed: Sep. 17, 2004
(86) PCT No.: PCT/CA04/01693

§ 371 Date: Mar. 3, 2006

Related U.S. Application Data
(60) Provisional application No. 60/505,244, filed on Sep. 23, 2003.

(51) Int. Cl. C12Q 1/48; C07C 245/02

(52) U.S. Cl. 435/15; 435/183; 534/11; 534/886

(57) ABSTRACT

The present invention relates to a method of identifying and evaluating chemical inhibitors of cathepsin S activity in whole cells. The present invention also relates to probes that are capable of forming irreversible adducts with cathepsin S at the active site. The probes used in the instant invention comprise radioactive functional groups to allow the detection of the irreversible cathepsin S-probe adducts.

### PRIORITY CLAIM

This application is a §371 National Stage Application of PCT/CA2004/001693, filed on Sep. 17, 2004, which claims priority from U.S. Provisional Application Ser. No. 60/505,244, filed on Sep. 23, 2003.

### BRIEF DESCRIPTION OF THE INVENTION

The present invention relates to a whole cell assay for identifying and evaluating inhibitors of cathepsin S, and to synthetic chemical probes which form irreversible adducts with cathepsin S at its active site.

### BACKGROUND OF THE INVENTION

Cathepsins belong to the papain superfamily of cysteine proteases. These proteases function in the normal physiological as well as pathological degradation of connective tissue. Cathepsins play a major role in intracellular protein degradation and turnover and remodeling. To date, a number of cathepsins have been identified and sequenced from a number of sources. These cathepsins are naturally found in a wide variety of tissues. For example, cathepsins B, F, H, L, K, S, W, and Z have been cloned.

Endocytic proteases, particularly cathepsins, have been identified as key regulatory molecules involved in the function of major histocompatibility (MHC) class II molecules in professional antigen-presenting cells (APC) (Weindl, H., et al, 2003, J Neuroimmn. 138:132-143). Cathepsin S is a lysosomal cysteine protease that is primarily expressed in macrophages, B cells and dendritic cells (Pauly, T. A., et al, 2003, Biochem. 42:(3203-3213). Cathepsin S plays a role in the generation of antigenic peptide from ingested complex proteins, and also controls maturation and transport of Class II MHC molecules. Class II MHC molecules consist of α/β heterodirners that are associated with a third glycoprotein known as invariant chain Ii, which functions to promote the correct folding of the MHC Class II molecule (Honey, K., et al, 2001, J. Biol. Chem., 276:22573-22578).

Cathepsin S plays a role in the stepwise degradation of the invariant chain Ii, as part of an essential process that allows the cell to present antigens on its surface. It has also been shown that cathepsin S is involved in the processing of the antigens themselves for presentation on MHC Class II molecules.

The involvement of cathepsin S in critical steps associated with antigen presentation suggests that inhibition of this protease may be useful for the treatment of diseases that are associated with elevated immune responses, such as asthma, organ transplant rejection, and various autoimmune disorders. Since cathepsin S has also been found in the lung, it has been suggested that it may play a role in the development of emphysema.

### SUMMARY OF THE INVENTION

In one embodiment, the present invention relates to a method for identifying a compound as an inhibitor of cathepsin S activity comprising the steps of
a) incubating eukaryotic host cells possessing endogenous cathepsin S activity with said compound;
b) adding a substrate to said eukaryotic host cells in the presence of said compound;
c) incubating said substrate in the presence of said compound;
d) stopping the reaction;
e) quantifying the amount of said substrate in complex with cathepsin S in said eukaryotic host cells; and
f) identifying said compound as an inhibitor of cathepsin S activity.

Another embodiment of the present invention relates to the use of substrates comprised of synthetic probes, which form irreversible adducts with cathepsin S at the active site via an electrophilic functionality of said probe. The present invention is further directed to synthetic probes labeled with a moieties selected from the group consisting of a radioactive functional group and a non-radioactive functional group. The present invention further includes a means of identifying a compound as an inhibitor of cathepsin S activity, whereby the ability of the compound to compete with the synthetic probe for the active site of cathepsin S is determined.

Unless otherwise defined, all technical and scientific terms used herein in their various grammatical forms have the same meaning as commonly understood by one of ordinary skill in the art to which this invention belongs. Although methods and materials similar to those described herein can be used in the practice or testing of the present invention, the preferred methods and materials are described below. In case of conflict, the present specification, including definitions, will control. In addition, the materials, methods, and examples are illustrative only and are not limiting.

Further features, objects and advantages of the present invention are apparent in the claims and the detailed description that follows. It should be understood, however, that the detailed description and the specific examples, while often indicating preferred aspects of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows the results of an autoradiographic analysis of human cathepsin S in peripheral human whole blood. White blood cells were purified by sedimentation after being labeled with N-[(1S)-1-[[[1-(2-diazoacetyl)butyl]amino]carbonyl]-3-methylbutyl]-4′-[¹²⁵I]-[1,1′-biphenyl]-4-carboxamide. Labeling of human cathepsin S was detected by autoradiography after separation by polyacrylamide gel electrophoresis (SDS-PAGE).

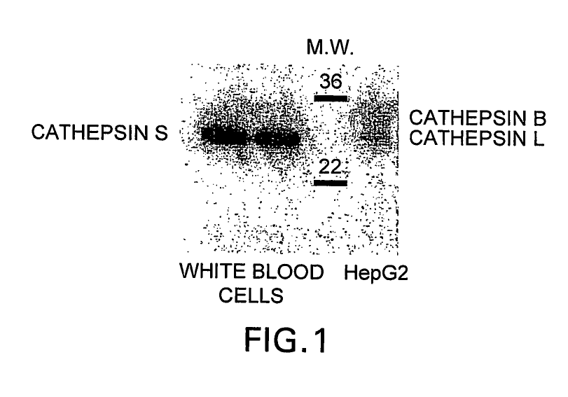

### DETAILED DESCRIPTION OF THE INVENTION

Cell-Based Assay

The present invention is directed to the development of a cell-based assay for identifying compounds as inhibitors of cathepsin S activity.

In a first embodiment of the present invention there is provided a cell-based assay for identifying a compound as an inhibitor of cathepsin S activity which comprises the steps of:
a) incubating eukaryotic host cells possessing endogenous cathepsin S activity with said compound;
b) adding a substrate to said eukaryotic host cells in the presence of said compound;
c) incubating said substrate in the presence of said compound;
d) stopping the reaction;
e) quantifying the amount of said substrate in complex with cathepsin S in said eukaryotic host cells; and
f) identifying said compound as an inhibitor of cathepsin S activity.

In another embodiment of the present invention, the eukaryotic host cells are peripheral human whole blood cells.

In another embodiment of the present invention, the incubation of the peripheral human whole blood cells with the compound occurs for a period of about 30 minutes to about three hours and at a temperature between 20° C. temperature to about 37° C.

In another embodiment of the present invention, the substrate comprises a synthetic probe that forms an irreversible adduct with cathepsin S at the active site via an electrophilic functionality of said probe.

In another embodiment of the present invention, the electrophilic functionality of the probe comprises ketones substituted in alpha with a leaving group.

In another embodiment of the present invention, the probe also comprises a functional group to allow the detection of the irreversible cathepsin S-probe adduct.

In another embodiment of the present invention, the functional group comprises a moiety selected from the group consisting of a radioactive functional group and a non-radioactive functional group.

In another embodiment of the present invention, the radioactive functional group is ¹²⁵Iodine, and the non-radioactive functional group is iodine.

In another embodiment of the present invention, the non-radioactive functional group is used to modulate the amount of radioactivity used in the method.

In another embodiment of the present invention, the incubation of the substrate in the presence of the compound occurs for a period of about 30 minutes to about three hours and at a temperature between 20° C. temperature to about 37° C.

In another embodiment of the present invention, the reaction is stopped by the addition of an irreversible inhibitor that acts by binding to the active site cysteine residue of cathepsin S.

In another embodiment of the present invention, the irreversible inhibitor is N-[(1S)-1-[[[1-(2-diazoacetyl)butyl]amino]carbonyl]-3-methylbutyl]-4′-iodo-[1,1′-biphenyl]-4-carboxamide.

In another embodiment of the present invention, the irreversible inhibitor is E64-D.

In another embodiment of the present invention, the amount of substrate in complex with cathepsin S is quantified based on measuring the radioactivity acquired by the peripheral human whole blood cells,.

In another embodiment of the present invention, a compound is identified as an inhibitor of cathepsin S activity based on its ability to compete with the substrate for the active site of cathepsin S in eukaryotic host cells.

The above-described assay method is explicitly directed to testing “a” compound, however it will be clear to a person skilled in the art that such a method can be adapted to testing multiple compounds, e.g., combinatorial libraries to determine if any member of such a collection is inhibitory to cathepsin S activity. Accordingly, the use of collections of compounds, or individual members of such collections is within the scope of this invention.

Synthetic Probes

The present invention is also directed to certain labeled compounds that are capable to binding irreversibly to the active site of cathepsin S. In particular, the present invention is directed to a compound of formula I: 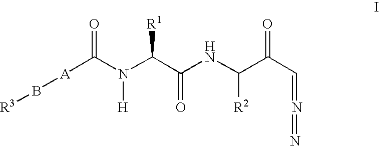
wherein
A and B are independently selected from 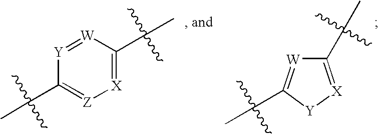
W, X, Y and Z are independently selected from CH, S, N or O;
R¹ is selected from C₁-C₁₀ alkyl, C₂-C₁₀ alkenyl, C₃-C₁₀ cycloalkyl, aralkyl or —(CR^{a}₂)ₜSO₂—;
wherein said groups are optionally substituted on the carbon or the sulfur with one to five substituents selected from halogen, C₁-C₁₀ alkyl, C₂-C₁₀ alkenyl, C₃-C₁₀ cycloalkyl, aryl or heterocyclyl;
R² is selected from C₁-C₁₀ alkyl, C₂-C₁₀ alkenyl, C₃-C₁₀ cycloalkyl, aryl, aralkyl, or heterocyclyl; wherein said alkyl, alkenyl, cycloalkyl, aryl, aralkyl and heterocyclyl groups are optionally substituted with one to five substituents selected from halogen, C₁-C₁₀ alkyl, C₂-C₁₀ alkenyl, C₃-C₁₀ cycloalkyl, aryl or heterocyclyl;
R³ is selected from ¹²⁵Iodine and Iodine;
Each R^{a} is independently selected from hydrogen and C₁-C₃ alkyl;
t is 0to 3;
or a pharmaceutically acceptable salt or stereoisomer thereof.

A further embodiment of the present invention is a compound of Formula I, or a pharmaceutically acceptable salt or stereoisomer thereof, as described above, wherein R¹ is selected from C₁-C₁₀ alkyl, C₃-C₁₀ cycloalkyl, or aralkyl;

wherein said alkyl, cycloalkyl and aralkyl groups are optionally substituted with one to five of halogen; and R² is selected from C₁-C₁₀ alkyl, C₃-C₁₀ cycloalkyl, or aralkyl; wherein said alkyl, cycloalkyl, and aralkyl groups are optionally substituted with one to five of halogen.

Specific examples of compounds of the instant invention include: N-[(1S)-1-[[[1-(2-diazoacetyl)butyl]amino]carbonyl]-3-methylbutyl]-4′-[¹²⁵I]-[1,1′-biphenyl]-4-carboxamide 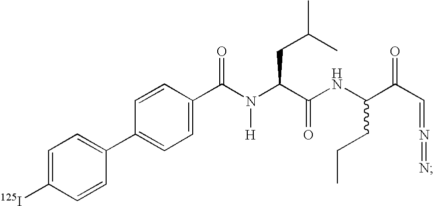
N-[(1S)-1-[[[1-(2-diazoacetyl)butyl]amino]carbonyl]-3-methylbutyl]-4′-iodo-[1,1′-biphenyl]-4-carboxamide 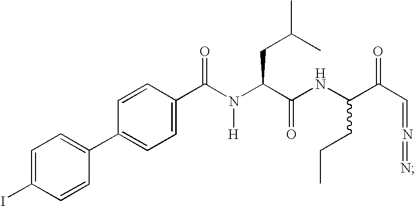
   or a pharmaceutically acceptable salt or stereoisomer thereof.
   Definitions

Unless defined otherwise, the scientific and technical terms and nomenclature used herein have the same meaning as commonly understood by a person of ordinary skill to which the invention pertains.

The term “endogenous” describes any naturally-occurring substance that is produced from within an organ or part. In the application herein, the cathepsin S protease is naturally produced by the eukaryotic host cells used in the whole cell assay.

The term “substrate” refers to a compound that is recognized by an enzyme and is a target for its activity. Such a compound can be synthesized, isolated and purified from any chemical or biological source, including recombinant DNA technology. In the application herein the enzyme is a protease and the substrates for detecting its enzymatic activity are comprised of a series of non-naturally occurring chemical compounds that have been designed to form irreversible adducts with cathepsin S at its active site. The substrates used herein on their own cannot be detected. It is therefore necessary to couple the substrates to indicator molecules, thereby enabling identification of the interaction of the substrate with cathepsin S. The coupled substrates and indicator molecules used herein are referred to as collectively as “synthetic probes”. The types of indicator molecules coupled to the synthetic probes include ¹²⁵Iodine. By assaying for the presence or absence of the synthetic probe, one can determine whether a potential inhibitor compound has successfully bound to the active site of cathepsin S, thereby displacing the synthetic probe from the binding site.

The term “adduct” refers to a chemical addition product, or more specifically, to a molecular entity that is formed by the direct combination of two separate molecular entities. The combination occurs in such a way that there is a change in connectivity but no loss of atoms from either of the separate entities that are combined.

The term “electrophilic functionality” refers to an assemblage of atoms that will en compass a partial or full positive charge or dipole than can form an adduct with molecules bearing a partial of full negative charge.

The term “ketones substituted in alpha with leaving group” refers to ketones in which the alpha carbon is substituted with an atom (or array of atoms) whose bond with the alpha carbon of the ketone is such that this atom (or array of atoms) can be displaced by with molecules bearing a partial of full negative charge.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted or named.

As used herein, “alkyl” is intended to include both branched and straight-chain aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, propyl, isopropyl, butyl, t-butyl, pentyl, hexyl, heptyl, octyl, nonyl, decyl, and so on. “Cycloalkyl” as used herein is intended to include non-aromatic cyclic hydrocarbon groups, having the specified number of carbon atoms, which may or may not be bridged or structurally constrained. Examples of such cycloalkyls include, but are not limited to, cyclopropyl, cyclobutyl, cyclopentyl, cyclohexyl, adamantyl, cyclooctyl, cycloheptyl, tetrahydro-naphthalene, methylenecylohexyl, and the like. As used herein, examples of “C₃-C₁₀ cycloalkyl” may include, but are not limited to:

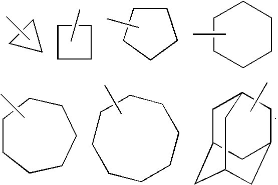

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to 4 non-aromatic carbon-carbon double bonds may be present. Thus, “C₂-C₆ alkenyl” means an alkenyl radical having from 2 to 6 carbon atoms. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated.

As used herein, “aryl” is intended to mean any stable monocyclic or bicyclic carbon ring of up to 7 atoms in each ring, wherein at least one ring is aromatic. Examples of such aryl elements include phenyl, naphthyl, indanyl, indanonyl, indenyl, biphenyl, tetralinyl, tetralonyl, fluorenonyl, phenanthryl, anthryl, acenaphthyl, tetrahydronaphthyl, and the like.

As appreciated by those of skill in the art, “halo” or “halogen” as used herein is intended to include chloro, fluoro, bromo and iodo.

The term “heteroaryl”, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzimidazolyl, benzodioxolyl, benzotriazolyl, benzothiofuranyl, benzothiazolyl, furanyl, thienyl, benzothienyl, benzofuranyl, benzoquinolinyl, imidazolyl, isoquinolinyl, oxazolyl, isoxazolyl, indolyl, pyrazinyl, pyridazinyl, pyridinyl, pyrimidinyl, pyrrolyl, quinolinyl, tetrahydronaphthyl, tetrahydroquinoline, and the like.

The term “heterocycle” or “heterocyclic” or “heterocyclyl”, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. “Heterocycle” or “heterocyclyl” therefore includes the above mentioned heteroaryls, as well as dihydro and tetrathydro analogs thereof. Further examples of “heterocyclyl” include, but are not limited to the following: azepanyl, azetidinyl, benzimidazolyl, benzodioxolyl, benzofuranyl, benzofurazanyl, benzopyranyl, benzopyrazolyl, benzotriazolyl, benzothiazolyl, benzothienyl, benzothiofuranyl, benzothiophenyl, benzothiopyranyl, benzoxazepinyl, benzoxazolyl, carbazolyl, carbolinyl, chromanyl, cinnolinyl, diazepanyl, diazapinonyl, dihydrobenzofuranyl, dihydrobenzofuryl, dihydrobenzoimidazolyl, dihydrobenzothienyl, dihydrobenzothiopyranyl, dihydrobenzothiopyranyl sulfone, dihydrobenzothiophenyl, dihydrobenzoxazolyl, dihydrocyclopentapyridinyl, dihydrofuranyl, dihydroimidazolyl, dihydroindolyl, dihydroisooxazolyl, dihydroisoquinolinyl, dihydroisothiazolyl, dihydrooxadiazolyl, dihydrooxazolyl, dihydropyrazinyl, dihycldropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dioxanyl, dioxidotetrahydrothienyl, dioxidothiomorpholinyl, furyl, furanyl, imidazolyl, imidazolinyl, imidazolidinyl, imidazothiazolyl, imidazopyridinyl, indazolyl, indolazinyl, indolinyl, indolyl, isobenzofuranyl, isochromanyl, isoindolyl, isoindolinyl, isoquinolinone, isoquinolyl, isothiazolyl, isothiazolidinyl, isoxazolinyl, isoxazolyl, methylenedioxybenzoyl, morpholinyl, naphthpyridinyl, oxadiazolyl, oxazolyl, oxazolinyl, oxetanyl, oxoazepirpinyl, oxadiazolyl, oxidothiomorpholinyl, oxodihydrophthalazinyl, oxodihydroindolyl, oxoimidazolidinyl, oxopiperazinyl, oxopiperdinyl, oxopyrrolidinyl, oxopyrimidinyl, oxopyrrolyl, oxotriazolyl, piperidyl, piperidinyl, piperazinyl, pyranyl, pyrazinyl, pyrazolyl, pyridazinyl, pyridinonyl, pyridopyridinyl, pyridazinyl, pyridyl, pyrimidinyl, pyrrolyl, pyrrolidinyl, quinazolinyl, quinolinyl, quinolyl, quinolinonyl, quinoxalinyl, tetrahydrocycloheptapyridinyl, tetrahydrofuranyl, tetrahydrofuryl, tetrahydroisoquinolinyl, tetrahydropyranyl, tetrahydroquinolinyl, tetrazolyl, tetrazolopyridyl, thiadiazolyl, thiazolyl, thiazolinyl, thienofuryl, thienyl, thiomorpholinyl, triazolyl, azetidinyl, 1,4-dioxanyl, hexahydroazepinyl, and the like.

As used herein, “aralkyl” is intended to mean an aryl moiety, as defined above, attached through a C₁-C₁₀ alkyl linker, where alkyl is defined above. Examples of aralkyls include, but are not limited to, benzyl, naphthylmethyl and phenylpropyl.

As used herein, the terms “substituted “C₁-C₁₀ alkyl” and “substituted C₂-C₁₀ alkenyl” are intended to include the branch or straight-chain alkyl group of the specified number of carbon atoms, wherein the carbon atoms may be substituted with 1 to 3 substituents selected from the group which includes, but is not limited to, halo, C₁-C₂₀ alkyl, CF₃, NH₂, N(C₁-C₆ alkyl)₂, NO₂, oxo, CN, N₃, —OH, —O(C₁-C₆ alkyl), C₃-C₁₀ cycloalkyl, C₂-C₆ alkenyl, C₂-C₆ alkynyl, (C₀-C₆ alkyl) S(O)₀₋₂—, (_{C}₀-C₆ alkyl)S(O)₀₋₂(C₀-C₆ alkyl)-, (C₀-C₆ alkyl)C(O)NH—, H₂N—C(NH)—,

—O(C₁-C₆ alkyl)CF₃, (C₀-C₆ alkyl)C(O)—, (C₀-C₆ alkyl)OC(O)—, (C₀-C₆ alkyl) O(C₁-C₆ alkyl)-, (C₀-C₆ alkyl)C(O)₁₋₂(C₀-C₆ alkyl)-, (C₀-C₆ alkyl)OC(O)NH—, aryl, aralkyl, heterocyclyl, heterocyclylalkyl, halo-aryl, halo-aralkyl, halo-heterocyclyl, halo-heterocyclylalkyl, cyano-aryl, cyano-aralkyl, cyano-heterocycle and cyano-heterocyclylalkyl.

As used herein, the terms “substituted C₃-C₁₀ cycloalkyl”, “substituted aryl”, and “substituted heterocyclyr”, are intended to include the cyclic group containing from 1 to 3 substituents in addition to the point of attachment to the rest of the compound. Preferably, the substituents are selected from the group which includes, but is not limited to, halo, C₁-C₂₀ alkyl, CF₃, NH₂, N(C₁-C₆ alkyl)₂, NO₂, oxo, CN, N₃, —OH, —O(C₁-C₆ alkyl), C₃-C₁₀ cycloalkyl, C₂-C₆ alkenyl, C₂-C₆ alkynyl, (C₀-C₆ alkyl) S(O)₀₋₂—, (C₀-C₆ alkyl)S(O)₀₋₂(C₀-C₆ alkyl)-, (C₀-C₆ alkyl)C(O)NH—, H₂N—C(NH)—, —O(C₁-C₆ alkyl)CF₃, (C₀-C₆ alkyl)C(O)—, (C₀-C₆ alkyl)OC(O)—, (C₀-C₆alkyl)O(C₁-C₆ alkyl)-, (C₀-₆ alkyl)C(O)₁₋₂(C₀-₆ alkyl), (C₀-C₆ alkyl)OC(O)NH—, aryl, aralkyl, heteroaryl, heterocyclylalkyl, halo-aryl, halo-aralkyl, halo-heterocyclyl, halo-heterocyclylalkyl, cyano-aryl, cyano-aralkyl, cyano-heterocyclyl and cyano-heterocyclylalkyl.

In an embodiment of this invention, R¹ is substituted or unsubstituted C₁-C₁₀ alkyl, substituted or unsubstituted C₂-C₁₀ alkenyl, or substituted or unsubstituted cycloalkyl. In another embodiment of this invention, R¹ is C₁-C₁₀ alkyl.

In an embodiment of this invention, R² is substituted or unsubstituted C₁-C₁₀ alkyl, substituted or unsubstituted C₂-C₁₀ alkenyl, or substituted or unsubstituted cycloalkyl. In another embodiment of this invention, R² is C₁-C₁₀ alkyl.

It is intended that the definition of any substituent or variable (e.g., R¹, R^{a}, etc.) at a particular location in a molecule be independent of its definitions elsewhere in that molecule. It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials.

### EXAMPLE 1

### Synthesis of Methyl N-[(4′-hydroxybiphenyl-4-yl)carbonyl]-L-leucinate

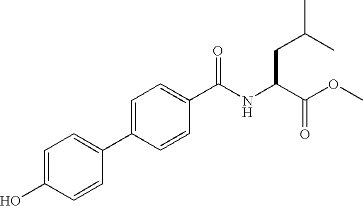

4′-Hydroxybiphenyl4-carboxylic acid (2.75 g, 12.9 mmoles) and L-leucine methylester hydrochloride (2.8 g, 15.5 mmoles) are suspended in dimethylformamide (20 mL) at room temperature. To the reaction mixture is added HATU (5.2 g, 13.7 mmoles) followed one minute later by triethylamine (7.2 mL, 51.6 mmoles). The reaction is stirred for one hour, diluted with ethyl acetate (200 mL) and 1 N hydrochloric acid (100 mL). The phases are separated and the organic phase washed with 0.1 N hydrochloric acid (100 mL) followed by water (100 mL) then brine (100 mL). The organic phase is dried over magnesium sulfate and concentrated under reduced pressure to afford the title compound in good purity.

### EXAMPLE 2

### Synthesis of Methyl N-[(4′-hydroxybiphenyl-4-yl)carbonyl]-L-leucylnorvalinate

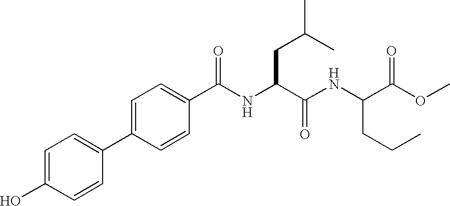

Methyl N-[(4′-hydroxybiphenyl-4-yl)carbonyl]-L-leucinate (2.78 g, 8.1 mmoles) is dissolved in 40 mL of a mixture of tetrahydrofuran, methanol and water to obtain a clear solution. Lithium hydroxyde monohydrate (855 mg, 20.4 mmoles) was added and the reaction mixture was stirred until the disappearance of the starting material by TLC. The reaction was quenched with 1N HCl until pH=1 (50 mL) and the aqueous phase extracted 3 times with dichloromethane (125 mL). The combined organic layers were dried over magnesium sulfate and concentrated under reduced pressure. The crude acid was dissolved in dimethylformamide (25 mL) along with norvalinemethylester hydrochloride (1.64 g, 9.8 mmoles). HATU (3.3 g, 8.6 mmoles) was added followed by triethylamine (4.5 mL, 33 mmoles) one minute later. The reaction was stirred for one hour, diluted with ethyl acetate (200 mL) and 1 N hydrochloric acid (100 mL). The phases are separated and the organic phase washed with 0.1 N hydrochloric acid (100 mL) followed by water (100 mL) then brine (100 mL). The organic phase is dried over magnesium sulfate and concentrated under reduced pressure to afford the title compound in very good purity after a swish in ether.

### EXAMPLE 3

### Synthesis of Methyl N-[(4′-{[(trifluoromethyl)sulfonyl]oxy}biphenyl-4-yl)carbonyl]-L-leucylnorvalinate

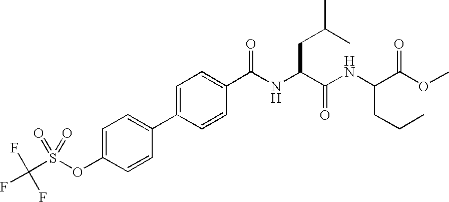

Methyl N-[(4′-hydroxybiphenyl-4-yl)carbonyl]-L-leucylnorvalinate (1.9 g, 4.3 mmoles) was dissolved in dichloromethane (20 mL) and cooled to −20° C. for the addition of triethylamine (1.8 mL, 13 mmoles). Trifluoromethanesulfonic anhydride (0.9 mL, 5.4 mmoles) was added to the reaction mixture over 2 minutes. Examination of the reaction progress by TLC after 10 minutes showed the consumption of all starting material. The reddish reaction mixture was poured onto a mixture of ether (100 mL) and saturated aqueous sodium bicarbonate (75 mL) in a separatory funnel. The phases were separated and the organic phase was successively washed with dilute aqueous sodium bicarbonate (100 mL), 1N hydrochloric acid (100 mL), water (100 mL) and brine (50 mL). The ethereal layer was dried over magnesium sulfate and concentrated under reduced pressure. The residue was purified by flash chromatography over silica gel using 50% Hexanes, 30% ethyl acetate and 20% dichloromethane to obtain the desired material.

### EXAMPLE 4

### Synthesis of Methyl N-{[4′-(trimethylstannyl)biphenyl-4-yl]carbonyl}-L-leucylnorvalinate

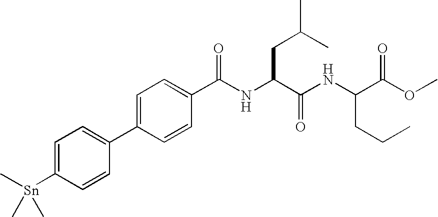

A suspension of Methyl N-[(4′-{[(trifluoromethyl)sulfonyl]oxy}biphenyl-4-yl)carbonyl]-L-leucylnorvalinate (1.9 g, 3.3 mmoles), 2,6-di-tert-butyl-4-methylphenol (few crystals) and lithium chloride (425 mg, 10 mmoles) in dioxane (30 mL) was degassed by three vacuum-Nitrogen flush cycles at room temperature. Hexamethylstannane (1.2 g, 3.7 mmoles) was added followed by palladium tetrakistriphenylphosphine (192 mg, 0.17 mmoles) and the reaction vessel immersed in a 98° C. oil bath for 3 hours. The mixture was poured onto a mixture of ether (100 mL) and saturated aqueous sodium bicarbonate (75 mL) in a separatory funnel. The phases were separated and the organic phase was successively washed with 0.1N hydrochloric acid (100 mL), saturated aqueous sodium bicarbonate (100 mL) and brine (50 mL). The ethereal layer was dried over magnesium sulfate and concentrated under reduced pressure. The residue was purified by flash chromatography over silica gel using a gradient from 70% Hexanes, 30% ethyl acetate to 50% Hexanes, 50% ethyl acetate to obtain the desired material.

### EXAMPLE 5

### Synthesis of N-{[4′-(trimethylstannyl)biphenyl-4-yl]carbonyl}-L-leucylnorvaline

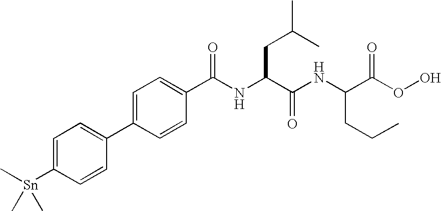

The Methyl N-{[4′-(trimethylstannyl)biphenyl-4yl]carbonyl}-L-leucylnorvalinate (1.2 g, 2.0 mmoles) is dissolved in 15 mL of a mixture of tetrahydrofuran, methanol and water to obtain a clear solution. Lithium hydroxyde hydrate (130 mg, 3.1 mmoles) was added and the reaction mixture was stirred for nine hours. The reaction was quenched with 1N HCl (20 mL) until pH=1 approximatively and the aqueous phase extracted 3 times with dichloromethane (75 mL). The combined organic layers were dried over magnesium sulfate and concentrated under reduced pressure to give the desired material in fair purity.

### EXAMPLE 6

### Synthesis of N-[(1S)-1-[[[1-(2-diazoacetyl)butyl]amino]carbonyl]-3-methylbutyl]-4′-iodo-[1,1′-biphenyl]-4-carboxamide

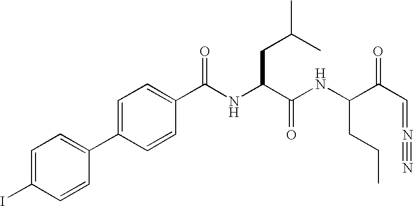

N-{[4′-(trimethylstannyl)biphenyl-4-yl]carbonyl}-L-leucylnorvaline (540 mg, 0.94 mmoles) in dichloromethane (10 mL) at room temperature was treated with an excess of iodine as solution in dichloromethane until the color stayed for 3 minutes. The reaction mixture was diluted with a mixture aqueous sodium bicarbonate (25 mL) and aqueous saturated sodium bisulfite until the system becomes colorless after shaking. The organic phase was dried over magnesium sulfate and concentrated under reduced pressure. The crude acid and N-methylmorpholine (0.25 ml, 2.3 mmoles) in tetrahydrofuran (10 mL) were cooled to 0° C. for the addition of iso-butyl chloroformate (0.14 mL, 1.1 mmoles) and stirred for 20 minutes. An excess of a diethylether solution of diazomethane was added and the reaction stirred at room temperature for 90 minutes and diluted with ether (75 mL) and water (75 mL). The phases are separated and the organic phase was successively washed with dilute aqueous sodium bicarbonate (50 mL), water (50 mL), brine (50 mL) and dried over magnesium sulfate. After concentration under reduced pressure, the residue was purified over silica gel using 50% hexanes, 50% ethyl acetate to afford the cold (not radioactive) desired material.

### EXAMPLE 7

### Synthesis of N-[(1S)-1-[[[1-(2-diazoacetyl)butyl]amino]carbonyl]-3-methylbutyl]-4′-trimethylstannyl-[1,1′-biphenyl]-4-carboxamide

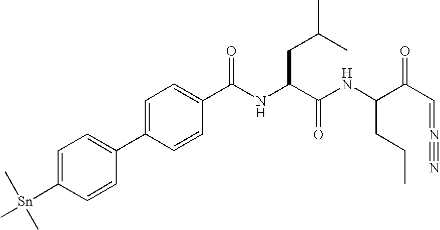

N-{[4′-(trimethylstannyl)biphenyl-4-yl]carbonyl}-L-leucylnorvaline (240 mg, 0.42) and N-methylmorpholine (0.075 mL, 0.6 mmoles) in tetrahydrofuran (4 mL) were cooled to 0° C. for the addition of iso-butyl chloroformate (0.06 mL, 0.5 mmoles) and stirred for 20 minutes. An excess of a diethylether solution of diazomethane was added and the reaction stirred at room temperature for 90 minutes and diluted with ether (50 mL) and water (50 mL). The phases are separated and the organic phase was successively washed with dilute aqueous sodium bicarbonate (30 mL), water (30 mL), brine (30 mL) and dried over magnesium sulfate. After concentration under reduced pressure, the residue was purified over silica gel using 50% hexanes, 50% ethyl acetate to afford the desired material.

### EXAMPLE 8

### Synthesis of N-[(1S)-1-[[[1-(2-diazoacetyl)butyl]amino]carbonyl]-3-methylbutyl]-4′-[¹²⁵I]-[1,1′-biphenyl]-4-carboxamide

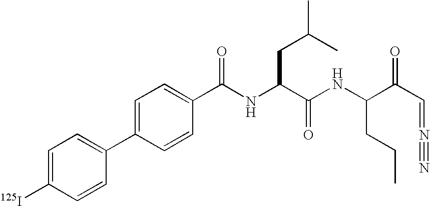

To a room temperature solution of N-[(1S)-1-[[[1-(2-diazoacetyl)butyl]amino]carbonyl]-3-methylbutyl]-4′-trimethylstannyl-[1,1′-biphenyl]-4-carboxamide (50 μL of a 4 mg/mL DMF solution, 0.33 μmol) and 150 μL of DMF was added carrier-free Na¹²⁵I (Draximage, 5 mCi in 0.1 mL of 0.1M NaOH) followed by chlorarmine-T (20 μL of a 10 mg/mL solution in 1:1 DMF:water, 0.7 μmol). The solution was stirred for 45 min, then quenched with 0.1N NaHSO₃ (40 μL, 4 μmol) and diluted with 150 μL of MeOH. The resulting solution was purified by RP HPLC (Zorbax C18 3.9×150 mm, 1 mL/min, MeOH/water containing 0.01% 2-mercaptoethanol) using the following gradient:
t=0′ 70%
t=5′ 70%
t=9′ 75% (linear gradient)
t=12′ 95% (linear gradient)
t=20′ 95%
t=21′ 70%
The two diastereomers of the title compound elute at 9′ and 10′. The fractions were combined to give 2.9 mCi of the title compound which was stored as a 0.5 μM solution in EtOH+0.01% 2-mercaptoethanol.

### EXAMPLE 9

### Synthesis of 4′-iodobiphenyl-4-carboxylic acid

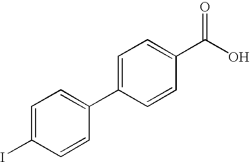

Biphenyl-4-carboxylic acid (13 g, 67 mmoles) in 135 mL of carbon tetrachloride at room temperature is treated with [bis(trifluoroacetoxy) iodo] benzene (32 g, 74 mmoles) followed by finely ground molecular iodine (17 g, 67 mmoles). After 1h, the reaction formed a gel so 70 mL of carbon tetrachloride were added and stirring resumed for 20 minutes. Solids were filtered and contained only the desired product. The compound was triturated in ether followed by leaving under high vacuum provided the desired product.

### EXAMPLE 10

### Synthesis of Methyl N-[(4′-iodobiphenyl-4-yl)carbonyl]-L-leucinate

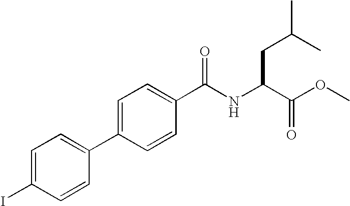

4′-Iodobiphenyl-4-carboxylic acid (15.8 g, 49 mmoles) and L-Leucine methyl ester hydrochloride (10.2 g, 56 mmoles) in dimethylformamide (100 mL) and tetrahydrofuran (100 mL) at room temperature were treated with HATU (19.8 g, 52 mmoles) followed by triethylamine (17 mL, 122 mmoles) one minute later. After 24 h, approximately half of the solvent system was removed under reduced pressure. The mixture is partitioned between half saturated aqueous sodium bicarbonate (300 mL) and ethyl acetate (500 mL). The phases were separated and the organic layer was washed with 1 N hydrochloric acid (250 mL), water (two 250 mL portions) then brine (200 mL). The solution is dried over magnesium sulfate and concentrated under reduced pressure to obtain a solid that is swished in ether with some ethyl acetate.

### EXAMPLE 11

### Synthesis of Methyl N-[(4′-iodobiphenyl-4-yl)carbonyl-L-leucylnorvalinate

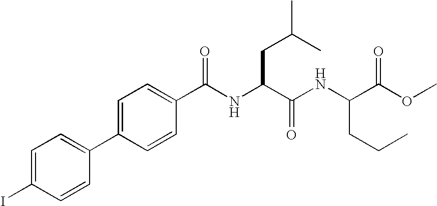

Methyl N-[(4′-iodobiphenyl-4-yl)carbonyl]L-leucinate (22.5 g, 50 mmoles) in 100 mL of tetrahydrofuran, 40 mL of methanol and 10 mL of water is treated with a suspension of lithium hydroxide monohydrate (3.15 g, 75 mmoles) in 20 mL of boiling water. The reaction was stirred until completion as judged by TLC. Reaction was carefully adjusted to pH 4 approximately by addition of 1 N HCl and most of the solvents were removed under reduced pressure. The concentrated suspension was diluted with dichloromethane (200 mL) and 1 N HCl (150 mL). The phases were separated and the aqueous phase was washed with dichloromethane (150 mL). The combined organic layers were dried over magnesium sulfate and concentrated under reduced pressure. The residue was dissolved in dimethylformamide (100 mL) and treated with racemic norvaline hydrochloride (8.6 g, 51 moles) followed by HATU (19.6 g, 51 mmoles). After one minute, triethylamine (17.5 mL, 125 mmoles) was added and the reaction stirred overnight. The reaction medium was diluted with ethyl acetate (300 mL), diethyl ether (100 mL) and 1N hydrochloric acid (300 mL). The phases were separated and the organic phase was washed with 0.1 N hydrochloric acid (200 mL), two 200-mL portions of water and brine (100 mL). The organic phase was dried over magnesium sulfate and concentrated under reduced pressure to obtain a solid which was purified by trituration in ether.

### Assays

Cats Whole Blood Enzme Occupancy Assay

Peripheral human whole blood is collected in heparin vacutainers. 1000 uL of blood in 1.5 mL Eppendorf tubes is treated with 2 uL of test compound (in DMSO) and incubated at room temperature for 30 min (for the ex vivo assay this step is omitted). 2 uL N-[(1S)-1-[[[1-(2-diazoacetyl)butyl]amino]carbonyl]-3-methylbutyl]-4′-[¹²⁵I]-[-1,1′-biphenyl]-4-carboxamide (0.5 uM stock in EtOH containing 0.01% of 2-mercaptoethanol, 2.5×10⁶ mCi/mmol, 2.5 uCi/tube) is added and the blood is incubated at room temperature for 30 min. The reaction is stopped by the addition of 2 uL of N-[(1S)-1-[[[1-(2-diazoacetyl)butyl]amino]carbonyl]-3-methylbutyl]-4′-iodo-[1,1′-biphenyl]-4-carboxamide (0.5 mM stock in DMSO). To this mixture, 120 uL of 3% w/v dextran (in PBS) is added and incubated at room temp for 60 min. 200 uL of the supernatant is removed and is centrifuged at 7000×g for 10 min (room temp or 4° C.). Remove and discard (approx 180 uL) supernatant and to the pellet add 100 uL SDS/PAGE sample buffer and heat to 95° C. for 10 min. Load 25 uL on 10-20% Tris-Glycine (Invitrogen/Novex) gel (12 well) and run (approx 90 min at 150 V). Dry gel and expose for 2-5 h at −70° C. using BioMax MS film (Kodak). Quantitate bands using a GS-800 Calibrated densitometer (BioRad) with QuantityOne software.

### Labeling of Cathepsin S in Ramos Cells with N-[(1S)-1-[[[1-(2-Diazoacetyl)Butyl]Amino]Carbonyl]-3-Methylbutyl]-4′-[¹²⁵I]-1,1′-Biphenyl]-4-Carboxamide

Materials

Complete Media

RPMI 1640 (Gibco #11875-093); 10% FBS heat inactivated; 10 mM Hepes; 1 mM Sodium Pyruvate; 100 U/mL penicillin; 100 ug/ml streptomycin
   Serum Free Media (SFM)
RPMI 1640 (Gibco #11875-093); 10 mM Hepes; 1 mM Sodium Pyruvate; 100 U/mL penicillin; 100 ug/mL streptomycin
N-[(1S)-1-[[[1-(2-diazoacetyl)butyl]amino]carbonyl]-3-methylbutyl]-4′-[¹²⁵I]-[1,1′-biphenyl]-4-carboxamide; (0.5 uM in ethanol+0.01% 2-mercaptoethanol)
   Sample Buffer (Laemmli)
Final buffer concentrations: 75 mM Tris-HCI (pH 6.8); 4.2% glycerol; 1.7% SDS 3.3% b-mercaptoethanol; bromophenol blue; Add 1 uM E-64 (Sigma #E-3132)
   Method

Ramos cells are resuspended at a density of 0.4×10E6 cells/ml 24 hours prior the experiment. After 24 hours, the cells are centrifuged and washed twice in serum free media then resuspended in the serum free media at a concentration of 1×10E7 cells/ml. 200 μL of cells are plated per well of a 96-well plate (Nunc). Compounds are added 100 fold concentrated in DMSO (2 uL) to obtained the following final concentrations.: 10 μM; 1 μM; 0.33 μM; 0.11 μM; 0.037 μM; 0.012 μM; 0.004 μM. Cells and compounds are incubated at 37° C.+5% CO₂ for 1 Hour then N -[(1S)-1-[[[1-(2-diazoacetyl)butyl]amino]carbonyl]-3-methylbutyl]-4′-[¹²⁵I]-[1,1′-biphenyl]-4-carboxamide is added at a final concentration of 1 nM. Stock solution of the probe is 0.5 μM in ethanol containing 0.01% 2-mercaptoethanol. The solution is diluted 5 fold in culture media and 5 uL of the solution is added to each well and cells are incubated at 37° C.+5% C₂ for 30 minutes then either 1 μM of E64d or 1 μM cold N-[(1S)-1-[[[1-(2-diazoacetyl)butyl]amino]carbonyl]-3-methylbutyl]4′-iodo-[1,1′-biphenyl]4-carboxamide is added into each well. The whole 96-well plate is then centrifuged at 300×g for 4 minutes, the supernatant removed from each well and 100 μL of sample buffer added. Samples are transferred in Eppendorf tubes and stored at −20° C. Samples are heated at 95° C. and loaded on a tris-glycine 10-20% PAGE. Gels are dried for 2 hours and exposed to a KODAK BIOMAX MS film for 2 to 3 hours at −80° C. then the film is scanned with the GS-800 calibrated imaging scanner (BioRad) and signal quantitated with Quantity One software by mean of volume analysis. % Inhibition is calculated relative to the DMSO control and IC₅₀ curve generated with Softmax Pro software (Molecular Devices).

What is claimed is:

1. A method for identifying a compound as an inhibitor of cathepsin S activity comprising the steps of
a) incubating eukaryotic host cells possessing endogenous cathepsin S activity with said compound;
b) adding a substrate to said eukaryotic host cells in the presence of said compound;
c) incubating said substrate in the presence of said compound;
d) stopping the reaction;
e) quantifying the amount of said substrate in complex with cathepsin S in said eukaryotic host cells; and
f) identifying said compound as an inhibitor of cathepsin S activity.

2. The method of claim 1, wherein said eukaryotic host cells are peripheral human whole blood cells.

3. The method of claim 2, wherein said incubation of said peripheral human whole blood cells with said compound occurs at a temperature between about 20° C. and about 37° C.

4. The method of claim 1, wherein said added substrate comprises a synthetic probe that forms an irreversible adduct with cathepsin S at the active site via an electrophilic functionality of said probe.

5. The method of claim 4, wherein said electrophilic functionality of said probe comprises ketones substituted in alpha with a leaving group.

6. The method of claim 4, wherein said probe also comprises a functional group to allow the detection of the irreversible cathepsin S-probe adduct.

7. The method of claim 6, wherein said functional group comprises a moiety selected from the group consisting of a radioactive functional group and a non-radioactive functional group.

8. The method of claim 7, wherein said radioactive functional group is ¹²⁵Iodine.

9. The method of claim 7, wherein said non-radioactive functional group is Iodine.

10. The method of claim 7, wherein said non-radioactive probe is used to modulate the amount of radioactivity used in said method.

11. The method of claim 1, wherein said incubation of said substrate in the presence of said compound occurs for a period of about 30 minutes to about 3 hours, and at a temperature between about 20° C. and about 37° C.

12. The method of claim 1, wherein the reaction is stopped by the addition of an irreversible inhibitor that acts by binding to the active site cysteine residue of cathepsin S.

13. The method of claim 12, wherein the irreversible inhibitor is N-[(1S)-1-[[[1-(2-diazoacetyl)butyl]amino]carbonyl]-3-methylbutyl]-4′-iodo-[1,1′-biphenyl]-4-carboxamide.

14. The method of claim 12, wherein the irreversible inhibitor is E-64-D.

15. The method of claim 1, wherein said compound is identified as an inhibitor of cathepsin S activity based on its ability to compete with said substrate for the active site of cathepsin S in said eukaryotic host cells.

16. A compound of Formula I 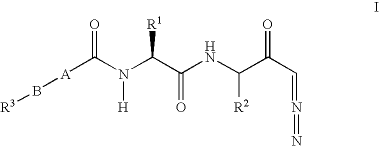
wherein:
A and B are independently selected from 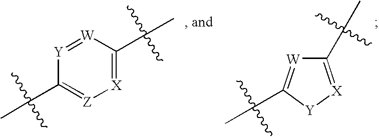
W, X, Y and Z are independently selected from CH, S, N or O;
R¹ is selected from C₁-C₁₀ alkyl, C₂-C₁₀ alkenyl, C₃-C₁₀ cycloalkyl, aralkyl or —(CR^{a}₂)ₜSO₂—;
wherein said groups are optionally substituted on the carbon or the sulfur with one to five substituents selected from halogen, C₁-C₁₀ alkyl, C₂-C₁₀ alkenyl, C₃-C₁₀ cycloalkyl, aryl or heterocyclyl;
R² is selected from C₁-C₁₀ alkyl, C₂-C₁₀ alkenyl, C₃-C₁₀ cycloalkyl, aryl, aralkyl, or heterocyclyl; wherein said alkyl, alkenyl, cycloalkyl, aryl, aralkyl and heterocyclyl groups are optionally substituted with one to five substituents selected from halogen, C₁-C₁₀ alkyl, C₂-C₁₀ alkenyl, C₃-C₁₀ cycloalkyl, aryl or heterocyclyl;
R³ is selected from ¹²⁵Iodine and Iodine;
Each R^{a} is independently selected from hydrogen and C₁-C₃ alkyl;
t is 0to 3;
or a pharmaceutically acceptable salt or stereoisomer thereof.

17. The compound according to claim 16, wherein
R¹ is selected from C₁-C₁₀ alkyl, C₃-C₁₀ cycloalkyl, or aralkyl; wherein said alkyl, cycloalkyl and aralkyl groups are optionally substituted with one to five halogen;
R² is selected from C₁-C₁₀ alkyl, C₃-C₁₀ cycloalkyl, or aralkyl; wherein said alkyl, cycloalkyl, and aralkyl groups are optionally substituted with one to five halogen;
or a pharmaceutically acceptable salt or stereoisomer thereof.

18. A compound which is 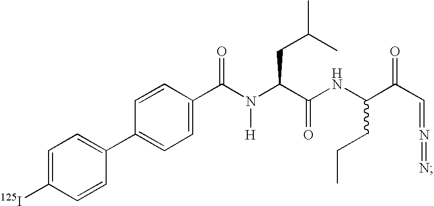
or a pharmaceutically acceptable salt or stereoisomer thereof.

19. A compound which is 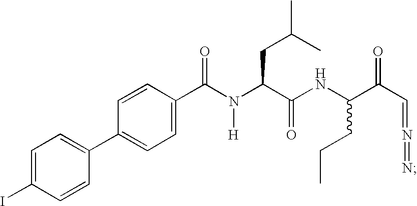
or a pharmaceutically acceptable salt or stereoisomer thereof.
